(19) United States
(12) Patent Application Publication
(10) Pub. No.: US 2002/0123045 A1
(43) Pub. Date: Sep. 5, 2002

(54) Plant transformation process with selection and early identification of germline events

(76) Inventors: Brian J. Martinell, Mt. Horeb, WI (US); Lori S. Julson, Lake Mills, WI (US); Venera Bouriakova, Madison, WI (US); Carol A. Emler, Mt. Horeb, WI (US); Dennis E. McCabe, Middleton, WI (US); Michael W. Petersen, Hillpoint, WI (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 09/683,399

(22) Filed: Dec. 21, 2001

Related U.S. Application Data
(60) Provisional application No. 60/258,137, filed on Dec. 22, 2000.

(51) Int. Cl. C12Q 1/68; A01H 5/00

(52) U.S. Cl. 435/6; 800/278; 800/279; 800/294

(57) ABSTRACT

A new method of identifying germline transformed plants early in the regeneration process is described. This method is based on the observation that if the roots of chimeric plants are transformed, then the plant is germline transformed. The method makes glyphosate selection more efficient and kanamycin selection practical.

### CROSS REFERENCE TO RELATED APPLICATIONS

This application claims priority to U.S. provisional filing No. 60/258,137, filed Dec. 22, 2000, herein incorporated by reference in its entirety.

### Background of Invention

The invention relates to methods for plant transformation and more particularly to methods providing an increased efficiency of selection of germline events.

Most plant transformation techniques rely on the introduction of foreign genes into individual cells of plant tissue maintained in tissue culture. Typically, one of the inserted foreign genes provides a means for selection of transformed cells when the transformed tissue is cultured in the presence of a selection agent. The selection agent kills all or substantially all of the non-transformed cells leaving the remaining transformed cells, which may then be regenerated into transgenic plants.

For a variety of reasons, this approach does not work well with all plants. This approach works best with plants that go through a callus stage during selection and then are regenerated back to whole plants. The difficulty is that many plants are resistant to regeneration from callus. Thus methods were developed to transform parts of the plant, such as the meristem, that are more amenable to regeneration of whole plants. Unfortunately, selection in such a system has been very difficult because the resulting transgenic plants are often chimeras. In other words, in every plant some cells are transformed and others are not. The difficulty is distinguishing which plants are transformed in their germline cells and are thus able to pass on the transformation event to their progeny. Early work used a visually screenable marker such as β-glucuronidase (GUS) to identify each plant that was transformed. The plants were then followed into the next generation to identify germline transformants.

U.S. Pat. No. 5,503,998 discusses early identification of germline transformation events. This method relies on screening of GUS patterns of expression in stem segments. Certain patterns were shown to correlate with germline transformation.

One disadvantage of using this method is the destruction of tissues that is necessary to assay for the enzyme. Additionally this method required precise sectioning of tissues and exacting microscopy for proper interpretation.

A method for selection of germline transformants using glyphosate selection has also been described (U.S. Pat. No. 5,914,451). It has been discovered that a certain percentage of plants that are difficult to root on glyphosate selection are indeed germline transformants. In most plants, transformation efficiencies are low; therefore, detecting all transformants is very important. Thus, if the plants that do not root easily on glyphosate selection are rooted off selection and could be tested for germline transformation, this would greatly increase the transformation efficiency.

It would also be advantageous to have other selectable markers available.

Unfortunately in the meristem transformation system, the standard selection agents available in callus culture are not very effective. For example, as many as 50% of the plants that root on kanamycin are not germline transformants.

A method of screening for germ line transformation has been developed that is less destructive to the plant than prior methods and only requires a positive or negative result rather than a complex classification scheme. It also allows for use of a wide variety of markers. Using this method of screening for germline transformants, kanamycin selection of meristem transformants is now efficient enough for practical use.

This method is based on the observation that the presence of the gene of interest in the roots indicates a germline transformation event. Although the presence of a selectable marker makes the process more efficient, this method allows for the direct assay of a gene of interest or any other nucleic acid sequence without the use of a selectable marker.

### SUMMARY OF INVENTION

The present invention provides a method for the rapid identification of germline transformed plants from the transformation of plant tissues that do not provide a sufficient number of germline events, such as meristematic tissue and cotyledonary tissue. In one aspect, the present invention provides a method for screening root tissue of putatively transformed plants for the presence of the nucleic acid introduced into the plant tissue to identify those plants likely to be germline transformed events.

In a further aspect of the invention, a method for increasing the efficiency of a transformation process to identify germline transformed events is provided involving rooting putatively transformed plants, which comprise a selected nucleic acid sequence of interest and a nucleic acid sequence encoding a selectable marker capable of identifying transformed plants containing the selectable marker nucleic acid sequence, in a root-inducing medium containing a selection agent corresponding to the selectable marker and assaying the roots of plants growing in the root inducing medium for the presence of the nucleic acid sequence of interest.

The present invention further provides a method of identifying germline transformed plants by transforming a meristem with a DNA construct, producing a plant shoot or cutting, inducing root formation, assaying the roots for the presence of the DNA construct, and selecting the germline transformants. The presence of the DNA construct in the roots is indicative of germline transformation and strongly correlated therewith.

The transformed plants may also be rooted in the presence of a selection agent, such as glyphosate or kanamycin.

The present invention also provides a method of transforming plants using kanamycin selection by transforming a plant meristem, selecting on kanamycin, assaying the roots for the presence of nptll, and identifying germline transformed plants.

### DETAILED DESCRIPTION

In order to provide a clear and consistent understanding of the specification and the claims, including the scope given to such terms, the following definitions are provided.

“Chimeric plants” are plants that are composed of tissues that are not genetically identical, i.e., the plants will have only a portion or fraction of their tissues transformed, whereas the remainder of the tissues are not genetically transformed. Particularly troublesome are plants that do not give rise to seeds containing the gene of interest (non-germline transformed).

“Germline transformation” occurs when the gene of interest is transformed into cells that give rise to pollen or ovule and thus into the seeds.

“Escapes” are traditionally plants that survive on selection even though they lack the selection marker gene. In the meristem transformation system, escapes are transformed plants, expressing at least the marker gene, that do not give rise to positive seeds (non-germline transformation). Although these plants are positive for the selectable markers or the genes of interest when measured by traditional methods, they have been difficult to distinguish from germline transformants without propagating them through the next generation.

In the invention, the genetic components are incorporated into a DNA composition such as a recombinant, double-stranded plasmid or vector molecule comprising at least one or more of the following types of genetic components: a promoter that functions in plant cells to cause the production of an RNA sequence, a structural DNA sequence that causes the production of an RNA sequence that encodes a product of agronomic utility, and a 3′ non-translated DNA sequence that functions in plant cells to cause the addition of polyadenylated nucleotides to the 3′ end of the RNA sequence. These are termed “plant expressible constructs.”

The vector may contain a number of genetic components to facilitate transformation of the plant cell or tissue and regulate expression of the desired gene (s).

In one embodiment, the genetic components are oriented so as to express a mRNA, which in one embodiment can be translated into a protein. The expression of a plant structural coding sequence (a gene, cDNA, synthetic DNA, or other DNA) that exists in double-stranded form involves transcription of messenger RNA (mRNA) from one strand of the DNA by RNA polymerase enzyme and subsequent processing of the mRNA primary transcript inside the nucleus. This processing involves a 3′ non-translated region that adds polyadenylated nucleotides to the 3′ end of the mRNA.

Means for preparing plasmids or vectors containing the desired genetic components are well known in the art. Vectors used to transform plants and methods of making those vectors are described in U.S. Pat. Nos. 4,971,908, 4,940,835, 4,769,061 and 4,757,011, the entirety of which are incorporated herein by reference. Vectors typically consist of a number of genetic components, including but not limited to regulatory elements such as promoters, leaders, introns, and terminator sequences. Regulatory elements are also referred to as cis- or trans-regulatory elements, depending on the proximity of the element to the sequences or gene(s) they control.

Transcription of DNA into mRNA is regulated by a region of DNA usually referred to as the “promoter.” The promoter region contains a sequence of bases that signals RNA polymerase to associate with the DNA and to initiate the transcription into mRNA using one of the DNA strands as a template to make a corresponding complementary strand of RNA.

A number of promoters that are active in plant cells have been described in the literature. Such promoters include, but are not limited to, the nopaline synthase (NOS) and octopine synthase (OCS) promoters, which are carried on tumor-inducing plasmids of Agrobacterium tumefaciens; the caulimovirus promoters such as the cauliflower mosaic virus (CaMV) 19S and 35S promoters and the figwort mosaic virus (FMV) 35S promoter; the enhanced CaMV35S promoter (e35S); and the light-inducible promoter from the small subunit of ribulose bisphosphate carboxylase (ssRUBISCO, a very abundant plant polypeptide). All of these promoters have been used to create various types of DNA constructs that have been expressed in plants. See, for example PCT publication WO 84/02913.

Promoter hybrids can also be constructed to enhance transcriptional activity (U.S. Pat. No. 5,106,739) or to combine desired transcriptional activity, inducibility, and tissue or developmental specificity. Promoters that function in plants are promoters that are inducible, viral, synthetic, constitutive as described (Poszkowski etal. , EMBO J., 3:2719, 1989; Odell etal., Nature, 313:810, 1985), and temporally regulated, spatially regulated, and spatio-temporally regulated (Chau etal. , Science, 244:174-181, 1989). Other promoters that are tissue-enhanced, tissue-specific, or developmentally regulated are also known in the art and envisioned to have utility in the practice of this invention. Promoters may be obtained from a variety of sources such as plants and plant DNA viruses and include, but are not limited to, the CaMV35S and FMV35S promoters and promoters isolated from plant genes such as ssRUBISCO genes. As described below, it is preferred that the particular promoter selected should be capable of causing sufficient expression to result in the production of an effective amount of the gene product of interest.

The promoters used in the DNA constructs (i.e., chimeric/recombinant plant genes) of the present invention may be modified, if desired, to affect their control characteristics. Promoters can be derived by means of ligation with operator regions, random or controlled mutagenesis, etc. Furthermore, the promoters may be altered to contain multiple “enhancer sequences” to assist in elevating gene expression. Examples of such enhancer sequences have been reported by Kay et al. (Science, 236:1299, 1987).

The mRNA produced by a DNA construct of the present invention may also contain a 5′ non-translated leader sequence. This sequence can be derived from the promoter selected to express the gene and can be specifically modified so as to increase translation of the mRNA. The 5′ non-translated regions can also be obtained from viral RNAs, from suitable eukaryotic genes, or from a synthetic gene sequence. Such “enhancer” sequences may be desirable to increase or alter the translational efficiency of the resultant mRNA. The present invention is not limited to constructs wherein the non-translated region is derived from both the 5′ non-translated sequence that accompanies the promoter sequence. Rather, the non-translated leader sequence can be derived from unrelated promoters or genes. (see, for example U.S. Pat. No. 5,362,865). Other genetic components that serve to enhance expression or affect transcription or translation of a gene are also envisioned as genetic components. The 3′ non-translated region of the chimeric constructs should contain a transcriptional terminator, or an element having equivalent function, and a polyadenylation signal, which functions in plants to cause the addition of polyadenylated nucleotides to the 3′ end of the RNA. Examples of suitable 3′ regions are (1) the 3′ transcribed, non-translated regions containing the polyadenylation signal of Agrobacterium tumor-inducing (Ti) plasmid genes, such as the nopaline synthase (NOS) gene, and (2) plant genes such as the soybean storage protein genes and the small subunit of the ribulose-1,5-bisphosphate carboxylase (ssRUBISCO) gene. An example of a preferred 3′ region is that from the ssRUBISCO E9 gene from pea (European Patent Application 385,962, herein incorporated by reference in its entirety).

Typically, DNA sequences located a few hundred base pairs downstream of the polyadenylation site serve to terminate transcription. The DNA sequences are referred to herein as transcription-termination regions. The regions are required for efficient polyadenylation of transcribed messenger RNA (mRNA) and are known as 3′ non-translated regions. RNA polymerase transcribes a coding DNA sequence through a site where polyadenylation occurs.

In one preferred embodiment, the vector contains a selectable, screenable, or scoreable marker gene. These genetic components are also referred to herein as functional genetic components, as they produce a product that serves a function in the identification of a transformed plant, or a product of desired utility. The DNA that serves as a selection device functions in a regenerable plant tissue to produce a compound that would confer upon the plant tissue resistance to an otherwise toxic compound. Genes of interest for use as a selectable, screenable, or scorable marker would include, but are not limited to, β-glucuronidase (GUS), green fluorescent protein (GFP), luciferase (LUX), antibiotic or herbicide tolerance genes. Examples of transposons and associated antibiotic resistance genes include the transposons Tns bla), Tn5 (npt II), Tn7 (dhfr); penicillins; kanamycin (and neomycin, G418, bleomycin); methotrexate (and trimethoprim); chloramphenicol; and tetracycline. Characteristics useful for selectable markers in plants have been outlined in a report on the use of microorganisms (Advisory Committee on Novel Foods and Processes, July 1994). These include (i) stringent selection with minimum number of nontransformed tissues; (ii) large numbers of independent transformation events with no significant interference with the regeneration; (iii) application to a large number of species; and (iv) availability of an assay to score the tissues for presence of the marker. As mentioned, several antibiotic resistance markers satisfy these criteria, including those resistant to kanamycin (nptII), hygromycin B (aph IV), and gentamycin (aac3 and aacC4).

A number of selectable marker genes are known in the art. Particularly preferred selectable marker genes for use in the present invention would include genes that confer resistance to compounds such as antibiotics, e.g., kanamycin (Dekeyser et al., Plant Physiol., 90:217-223,1989), and herbicides, e.g., glyphosate (Della-Cioppa et al., Bio/Technology, 5:579-584, 1987). Other selection devices can also be implemented and would still fall within the scope of the present invention.

The present invention can be used with any suitable plant transformation plasmid or vector containing a selectable or screenable marker and associated regulatory elements as described, along with one or more nucleic acids expressed in a manner sufficient to confer a particular desirable trait of agronomic utility. Examples of suitable structural trait genes of interest envisioned by the present invention would include, but are not limited to, genes for insect or pest tolerance, such as B. thuringienses genes; herbicide tolerance, such as genes for glyphosate resistance; genes for quality improvements such as nutritional enhancements, vaccines, protein production, oil quality enhancement; yield, such as biomass increases, source or sink enhancements, sugar increases; environmental or stress tolerances, such as drought or salt or water or cold tolerances; or any desirable changes in plant physiology, growth, development, morphology, or plant product(s). The gene of interest could also be present without a selectable or screenable marker. The current invention makes this more practical than previous methods allowed. The gene of interest and the selectable marker genes may also be present on separate T-DNAs (U.S. Pat. No. 5,731,1 9).

Alternatively, the DNA coding sequences can affect these phenotypes by encoding a non-translatable RNA molecule that causes the targeted inhibition of expression of an endogenous gene, for example via. antisense- or cosuppression-mediated mechanisms (see, for example, Bird et al., Biotech Gen. Engin. Rev., 9:207-227, 1991). The RNA could also be a catalytic RNA molecule (i.e., a ribozyme) engineered cleave a desired endogenous mRNA product (see, for example, Gibson and Shillitoe, Mol. Biotech., 7:125-137, 1997). Thus, any gene that produces a protein or mRNA that expresses a phenotype or morphology change of interest is useful for the practice of the present invention.

Exemplary nucleic acids that may be introduced by the methods encompassed by the present invention include, for example, DNA sequences or genes from another species, or even genes or sequences that originate with or are present in the same species but are incorporated into recipient cells by genetic engineering methods rather than classical reproduction or breeding techniques. However, the term exogenous is also intended to refer to genes that are not normally present in the cell being transformed or to genes that are not present in the form, structure, etc., as found in the transforming DNA segment or to genes that are normally present but a different expression is desirable. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the plant cell, DNA from another plant, DNA from a different organism, or a DNA generated externally, such as a DNA sequence containing an antisense message of a gene, or a DNA sequence encoding a synthetic or modified version of a gene.

In light of this disclosure, numerous other possible selectable or screenable marker genes, regulatory elements, and other sequences of interest will be apparent to those of skill in the art. Therefore, the foregoing discussion is intended to be exemplary rather than exhaustive.

To fully understand the advantages of the present method, it is helpful to appreciate certain considerations about the nature of meristem-based transformation of plants. Meristems are perpetually embryonic regions of cells and include the shoot apical meristems, cotyledons, hypocotyls, and the root meristems. Meristem-based transformation, either with Agrobacterium-mediated methods or with particle-mediated methods, results in chimeric plants, in which some, but not all, of the tissues have been transformed with the introduced DNA. Even with current selection methods, it is difficult to totally select for germline transformed plants because the efficiency of selection in a meristem-based system is much less than in a cell culture system. Without selection, most of the transformants do not result in germline transformation. With glyphosate selection, approximately 90% of the rooted plants are germine transformed, whereas with kanamycin selection only approximately 50% of the rooted plants are germine transformed. Any transformation method that chimeric plants would be applicable to the current invention.

It is not necessary to use the early germline identification process described here to achieve a germline transformed plant. It is possible to regenerate all plants recovered from the treated tissue, sexually propagate all the plants, and assay the progeny. The drawback to this approach is that most of the effort in the regeneration and propagation process will be wasted on the non-germline transformation events. The present invention helps to avoid that waste and thereby assists in the efficient creation of lines of genetically transformed plants.

This method is also useful because the heterologous DNA construct need not have any useful function. It can be assayed solely for its presence in the genome by PCR. However, the heterologous DNA construct will usually contain a gene of interest that confers a desired trait or a marker for successful transformation on the transformed plant. As known to those of ordinary skill in the art and discussed previously, such constructs will also contain appropriate flanking regulatory sequences suitable for expression of the foreign gene in a plant cell, such as a promoter sequence capable of initiating transcription and a translational terminator to terminate translation of a message if protein synthesis is desired. The transforming heterologous DNA construct may also include a marker gene. The marker gene can be a selectable marker, such as genes that confer resistance to glyphosate or kanamycin, or it can be a marker gene that can be assayed easily, such as GIJS. Once the meristem tissue is transformed and shoots are generated, roots can be induced from those shoots using standard rooting media known to those skilled in the art. Roots can be induced either in the presence or absence of selection agents. Rooting on selection helps to reduce the number of plants to be screened. However, it has been found that there are a number of germline transformants that resist rooting on glyphosate selection. In this case, roots can be induced in the absence of selection and then the roots tested for the presence of the gene of interest. This is particularly useful for genetic constructs that yield low transformation efficiencies.

There are many methods available for screening directly or indirectly for the gene of interest or the marker gene or fragments thereof and all are well known to those of skill in the art. They include, but are riot limited to, direct visualization of the DNA sequence through PCR, RT-PCR, or Southern blotting. Indirectly, the protein produced can be visualized through the use of an ELISA, fluorescent in situ hybridization (FISH), or histochemical or fluorescent staining (in the case of GFP or GUS). If the protein is an enzyme, it can be assayed in a variety of ways, including, but not limited to, spectrophotometric assays.

This method of germline identification can also be useful for the method development of new selection agents. The method allows for the testing of the efficiency of selection quickly and easily.

### EXAMPLES

The following examples further illustrate the present invention. They are in no way to be construed as a limitation in scope and meaning of the claims.

### Example 1

Transformed soybean plants were produced either by particle acceleration device transformation using glyphosate or kanamycin selection or by Agrobacterium-mediated transformation using kanamycin selection. Transformation was done with standard genetic constructs as described previously containing various genes of interest. Glyphosate selection was done on plants that were transformed with EPSPS synthase (a gene conferring tolerance to a glyphosate-containing herbicide), and kanamycin selection was done on plants that were transformed with nptII.

Soybeans were transformed by particle acceleration device essentially as described in U.S. Pat. No. 5,914,451 and selected on either glyphosate or kanamycin. Media formulations may be found in the cited references or in the media table (Table 1). Embryonic axes were excised from seeds germinated in liquid bean germination medium (BGM) overnight at 20° C. in the dark. The primary leaf tissue was carefully removed to expose the meristematic region.

Glyphosate Selection:

Explants were plated on Woody Plant Media (WPM) (McCown & Lloyd, Proc. International Plant Propagation Soc., 30:421, 1981) containing 2% sucrose, 200 mg/L carbenicillin, and 60 mg/L benomyl and incubated overnight at 15° C., dark.

A bead preparation for coating the blasting sheets was prepared as follows. One to five μL DNA (1 mg/mL) was added to 100 μL of 0.1 M spermidine. The spermidine/DNA solution was transferred to a vessel containing 10-20 mg of 0.82 or 0.95 μm beads and vortexed completely. One hundred μL 10% CaCl₂ was added dropwise with continuous vortexing. The mixture was allowed to stand for 10 minutes, during which time precipitation occurred. The supernatant was removed, and the DNA/gold precipitate was resuspended in 19 mL 100% ethanol plus 1 ml of sterile distilled water. A 320 μL aliquot of the bead preparation was used to coat each blasting sheet. For glyphosate selection, the DNA contained the gene for EPSPS synthase that confers resistance to glyphosate.

Explants were transferred to target medium (8% low viscosity carboxymethylcellulose, 2% medium viscosity carboxymethylcellulose, 0.4% washed agar) with the meristems facing up. The explants were bombarded once, using an electric discharge particle mediated gene delivery instrument. Following bombardment, explants were transferred to WPM media as listed above plus 75 μM glyphosate.

Shoots were cut 3-6 weeks post-bombardment and placed on BRM rooting media containing 25 to 40 μM glyphosate. Shoots that did not root after 3 weeks were placed on BRM without selection for an additional 2-3 weeks. Root samples were taken from shoots that rooted off-selection.

Kanamycin Selection:

Explants were plated on WPM media without selection, placed at 15° C., dark overnight and then moved to 4° C. for 3 days. One day prior to blast, explants were placed on OR medium and incubated overnight at room temperature in the dark. OR medium is MS medium, as modified by Barwale et al. (Planta 167:473-481, 1986) plus 3 mg/L BAP, 0.037 mg/L NAA, 200 mg/L carbenicillin, 62.5 mg/L cefotaxime, and 60 mg/L benomyl.

A bead preparation for coating the blasting sheets was prepared as above. For kanamycin selection, the DNA contained the nptil gene, which confers resistance to kanamycin.

Explants were transferred to target medium (8% low viscosity carboxymethylcellulose, 2% medium viscosity carboxymethylcellulose, 0.4% washed agar) with the meristems facing up. The explants were bombarded once, using an electric discharge particle mediated gene delivery instrument. Following bombardment, explants were transferred to WPM media as mentioned above with glyphosate replaced by 300 mg/L kanamycin nitrate. Explants were incubated at 25-28° C. with either a 16 hour light/8 hour dark or 18 hour light/6 hour dark photoperiod.

Shoots were cut 3-6 weeks post-bombardment and placed on BRM rooting media containing 175 mg/L kanamycin nitrate.

R1 seed was collected and crushed for analysis.

For Agrobacterium transformation, soybean seeds were soaked in sterile distilled water (SDW) for three minutes at room temperature, drained, and left moist for 2 hours. BGM medium was added after two hours to 2-3 times the depth of the seed volume and incubated at room temperature in the dark for 6 to 11 hours.

Seed axis embryos were recovered from germinated seed. Seed coats, cotyledons and primary leaves were removed, Freshly excised explants were placed into sterile petri dishes with SDW and rinsed 3 times with SDW once excision was complete.

Using a sterile forceps, each explant was wounded with a single poke from a #4 flat shader tattooing needle. The tattoo needle was oriented such that 3 of the 4 needles lined up with the centers of the meristems. Wounded explants were placed in a deep petri plate with fresh SDW. Once wounding was complete, all SDW was removed.

To each petri dish, ˜20-25 mL of Agrobacterium induced with 0.2 mM acetosryingone, 1.0 μM galacturonic acid and 0.25 mg/L GA₃ was added to completely cover explants. Explants were inoculated for 1 hr while shaking on an orbital shaker at 90 RPM.

Following inoculation, all Agrobacterium was removed. Explants were then transferred to co-culture plates containing 1 piece of autoclaved Whatman grade 1 filter paper and X mL of co-culture media); where X was 3 mL for 100, 5 mL for 200, 7 mL for 300 explants per plate. Co-culture media consists of 1/10 Gamborg's B5 media containing 1.68 mg/L BAP, 3.9 g/L MES and inducers as listed above (Gamborg et al., Exp. Cell Res., 50:151-158, 1968). Explants were gently spread evenly across the plate. All plates were placed in a dark box and incubated at 23° C. for 3 days.

After 3 days of co-culture, all explants were transferred to OR plus 225 mg/L kanamycin nitrate and incubated at 23° C. in the dark for 3 days. Elongating explants were then transferred to WPM plus 225 mg/L kanamycin nitrate and incubated at 28° C., 16 hr light/8 hr dark photoperiod. A transfer to fresh WPM plus 225 mg/L kanamycin nitrate was made after two weeks.

Shoots were present 3-4 weeks post-inoculation. All elongated shoots greater than 1 cm were cut and placed on BRIM plus 75 mg/L kanamycin nitrate. Shoots were monitored for root growth for one month. Rooted shoots were placed in a fresh plantcon of BRM or BRM plus kanamycin nitrate and sent to the greenhouse.

All plants were sampled for ELISA or PCR to detect the presence of the kanamycin selection gene.

Table 1—Media Bean Germination Media (BGM 2.5%)

Compound: Quantity Per Liter

BT STOCK#110 mL

BT STOCK #210 mL

BT STOCK #33 mL

BT STOCK #43 mL

BT STOCK #51 mL

SUCROSE 25 g

Adjust to pH 5.8.

DISPENSED IN 1 LITER MEDIA BOTTLES, AUTOCLAVED

ADDITIONS PRIOR TO USE: PER 1 L

CEFOTAXIME (50 mg/mL) 2.5 mL

FUNGICIDE STOCK 3 mL

BT STOCK FOR BEAN GERMINATION MEDIUM Make and store each stock individually. Dissolve each chemical thoroughly in the order listed before adding the next. Adjust volume of each stock accordingly. Store at 4° C.

BT stock 1 (1 liter)

KNO₃50.5 g

NH₄NO₃24.0 g

MgSO₄7H₂O 49.3 g

KH₂PO₄ 2.7 g

Bt Stock 2 (1 liter)

CaCl₂*2H₂ O 17.6 g

Bt Stock 3 (1 liter)

H₃BO₃0.62 g

MnSO₄₋H₂O 1.69 g

ZnSO₄−7H₂O 0.86 g

KI 0.083 g

NaMoO ₄−2H₂O 0.072 g

CuSO₄-5H₂O0.25 mL of 1.0 mg/mL stock

CoCl₄-6H₂O 0.25 mL of 1.0 mg/mL stock

Bt Stock 4 (1 liter)

Na₂ EDTA 1.116 g

FeSO₄7H₂O 0.834 g

Bt Stock 5 (500 mL) Store in a foil wrapped container

Thiamine-HCl 0.67 g

Nicotinic Acid 0.25 g

Pyridoxine-HCl 0.41 g

BRM MEDIA (for 4L)

MS Salts 8.6 g

Myo-Inositol (Cell Culture Grade) 0.40 g

Soybean Rooting Media Vitamin Stock 8 mL

L-Cysteine (10 mg/mL) 40 mL

Sucrose (Ultra Pure) 120 g

pH 5.8

Washed Agar 32 g

Additions After Autoclaving:

BRM Hormone Stock 20.0 mL

Ticarcillin/clavulanic acid (100 mg/mL Ticarcillin) 4.0 mL

Soy Tissue Culture Hormone Pre-Mixes

OR Pre-mixed Hormones

Use 10.0 mL per liter. Store dark at 4° C.

Amount for 1 liter

6.0 mL BAP (0.5 mg/mL)

0.037 mL NAA (1.0 mg/mL)

3.96 mL SDW

WPM Pre-mixed Hormones

Use 10.0 mL per liter

Amount for 1 liter

0.080 mL BAP (0.5 mg/mL)

9.92 mL SDW Store dark at 4° C.

BRM Hormone Stock Amount for 1 liter

6.0 mL IAA (0.033 mg/mL)

4.0 mL SDW Store dark at 4° C.

Vitamin Stock for Soybean Rooting Media (1 Liter)

Glycine 1.0 g

Nicotinic Acid 0.25 g

Pyridoxine HCl 0.25 g

Thiamine HCl 0.05 g

Dissolve one ingredient at a time, bring to volume, store in foil-covered bottle in refrigerator for no more than one month.

Fungicide Stock (100 mL)

chlorothalonile (75% WP) 1.0 g

benomyl (50% WP) 1.0 g

captan (50%WP) 1.0 g

Add to 100 mL of sterile distilled water.

Shake well before using.

Store 4° C. dark for no more than one week.

### Example 2

Root samples from RO plants were taken from tissue culture. Two main roots sampled per plant, approximately 1 cm each. Roots were assayed by CP4 dipstick ELISA or NPTII PCR.

R1 tissue was collected from dry seed shavings or seedling leaf tissue. Germline status was determined by CP4 dipstick ELISA or NPTII ELISA or NPTII PCR.

The CP4 dipstick ELISA was performed using CP4 dipsticks from Strategic Diagnostic Inc. (Newark, De.). Plant samples were collected in a microfuge tube and snap frozen in liquid nitrogen and then stored at −80 C. The sample was then ground in 500 μL of buffer. Buffers used were Leaf sample: 1× PBS, 0.5% Tween-20; Root sample: 1× PBS, 1% BSA, 1% Tween-20, 0.5% PVP; and Seed sample: 1× PBS, 1% BSA, 1% Tween-20, 0.5% PVP. After sample is extracted, one CP4 dipstick is put into tube and incubated at room temperature for 10-15 min. A negative result is one line near the top of the dipstick, and a positive result is two lines.

Qualitative NPTII ELISA is performed using a kit from AGDIA Inc. (Elkhard, Ind.).

Root Standard PCR Protocol

Stocks: Final Conc.

10× Reaction Buffer

500 mM KCl 50 mM KCl

100 mM Tris-HCl (pH9.0 at 25° C.) 10 mM Tris-HCl

1.0% Triton X-1000.1% Triton X-100

15 mM MgCl₂ 1.5 mM MgCl₂

10× dNTP's 0.2 mM of each dNTP

2 mM dATP, dTTP, dGTP, dCTP

50× Primer Stock (primers from Gibco BRL, Gaithersburg, Md.)

100 ng/μL (approx. 10 μM or 10 pmol) 2 ng/μL or 0.2 μM

Taq Polymerase (Gibco BRL)

5U/μL 0.02U/μL

Reaction. for Tubulin (Endogenous).

X μL DNA (20 μL out of 100 μL), 5 μL 10× Reaction Buffer, 5 μL 10× dNTP's, 2 μL 50× Primer DR 126, 2 μL 50× Primer DR 125, 0.2 μL Taq Polymerase, and X μL Sterile Distilled Water (to make total volume of 50 μL).

Reaction:for Kanamycin (NPTII):

X μL DNA (20 μL out of 100 μL), 5 μL 10× Reaction Buffer, 5 μL 10× dNTP's, 2 μL 50× Primer DR 35, 2 μL 50× Primer DR 39, 2 μL 50× PrimerJE 007, 2 μL 50× PrimerJE 008, 0.2 μL Taq Polymerase, X μL Sterile Distilled Water (to make total volume of 50 μL).

Before adding the DNA to the reaction incubate at 55° C. for 5 min. Combine components in 0.7 mL slick seal tube and overlay with 1 drop of mineral oil (approximately 10 μL). Run reaction in Perkin/Elmer/Cetus DNA thermocycler for 96° C. for 3 min., 96° C. for 1 min., 55° C. for 1 min. 15 sec., 72° C. for 1 min., repeat steps 2-4 for 30 total cycles, then 72° C. for 10 min., and 4° C. soak until ready to remove. Run 10 μL of sample in a 1.2% TBE gel for approximately 1 hour and stain with ethidium bromide.

Tables 2 and 3 show the first analysis of R1 plants from multiple constructs, indicating 75-100% correlation with plants rooting on glyphosate and germline transformation. Kanamycin selection had less of a correlation at 67%. Soybeans were transformed with the particle acceleration device with glyphosate or kanamycin selection.

[t3]

TABLE 2

| | Total Rooted | Total | % Rooting-Germline |
|---|---|---|---|
| Rooting Media | Plants | Germline | Correlation |
| BRM + 25 uM gly | 4 | 3 | 75% |
| BRM + 25 uM gly | 8 | 7 | 88% |
| BRM + 25 uM gly | 8 | 7 | 88% |
| BRM + 25 uM gly | 3 | 3 | 100% |
| BRM + 175 ppm kan | 3 | 2 | 67% |

Table 3 verifies the rooting on selection to germline transformation correlation seen in Table 2 when looking at multiple constructs rooted on either 25 or 40 μM glyphosate.

[t4]

TABLE 3

| | Total Rooted | Total | % Rooting-Germline |
|---|---|---|---|
| Rooting Media | Plants | Germline | Correlation |
| BRM + 25 uM gly | 47 | 38 | 81% |
| BRM + 40 uM gly | 45 | 38 | 84% |

A germline transformation correlation to positive assays done on root tissue of glyphosate or kanamycin rooted plants across multiple constructs also exists. Table 4 shows the correlation between root assays and germline transformed plants for both glyphosate and kanamycin selected plants. Plants on kanamycin were transformed via Agrobacterium transformation as described above and include 100 mg/L timetin (ticarcillin and clavulanic acid) in the rooting media.

TABLE 4

| | Total | Total Rooted | Total Root | % Root |
|---|---|---|---|---|
| | Selection | Plants with | Assay | Assay |
| | Rooted | Positive | Positive | Germline |
| Rooting Media | Plants | Root Assay | Germlines | Correlation |
| BRM + 25 uM gly | 34 | 34 | 25 | 74% |
| BRM + 75 mg/ | 62 | 25 | 16 | 70% |
| L kan + 100 mg/ | | | | |
| L Tim | | | | |

Utilizing the root assay-germline correlation seen in Table 4, root assays can be used to capture those plants that only root once removed from glyphosate selection but are still germline transformed. Of 124 total plants rooted off selection, 26 were positive with the root assay. Of those, 22 were germline positive, for an 85% correlation of root assay positive with germline. Eighteen percent of plants rooted off-selection were germline positive.

### Example 3

Transformed cotton plants were produced by particle acceleration device transformation as described in McCabe and Martinell, Biotechnology 11:596-598, 1993. Cotton seed was surface sterilized by soaking three minutes in 2.5% sodium hypochlorite. Seeds were rinsed in sterile distilled water, then soaked for an additional 24 hours at 28° C. in a fungicide suspension containing 30 mg/L each of captan, and benomyl and 45 mg/L of chlorothalonile plus 125 mg/L cefotaxime and 200 mg/L carbenicillin. Following surface sterilization, the seed was drained. Embryonic axes were removed from germinated seed and dissected to expose the meristem. The axes were then laid on modified MS medium containing 3 mg/L BAP (Barwale et al., 1986) and incubated overnight in the dark. These explants were then oriented so their meristems would be accessible to bombardment. Following bombardment, the axes were replated on the modified MS medium plus BAP for an additional 24 hours at 28° C. in darkness. After this healing period, the explants were transferred to plantcons containing WPM with 20 g/L glucose instead of sucrose. The axes were allowed to develop at 28° C. under a 16-hour phiotoperiod.

DNA on gold beads was prepared as described for soybean. In the case of cotton, the DNA contained the gene for GUS.

Cuttings were taken from greenhouse plants and were planted in a standard rooting mix and placed on a mist bench with heat coils. It was critical to force new adventitious roots, as the original roots were not transformed and did not represent the true germline status of the RO mericlonal plant. Roots were tested for GUS expression.

Results of petiole cross-sections and pollen assays (as described in McCabe and Martinell, 1993) correlate to germline transformed plants. Data show a strong correlation between root assays and petiole cross sections/pollen assays. Of 22 plants tested, 20 had matching results, showing a 91% correlation. Root assays can be used to determine germline transformation potential at a much earlier stage in development than previous methods.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the invention has been described in detail for the purpose of illustration, it is understood that such detail is solely for that purpose, and variations can be made therein by those skilled in the art without departing from the spirit and scope of the invention which is defined by the following claims.

1. A method for early identification of germline transformed plants comprising the steps of:
(a) transforming meristematic or cotyledonary tissue with a plant expressible construct comprising at least one nucleic acid sequence encoding a protein to obtain transformed plant tissue;
(b) producing a shoot from the transformed plant tissue;
(c) growing roots from the shoot;
(d) assaying the roots for the presence of the nucleic acid sequence; and
(e) identifying roots that assay positive for said at least one nucleic acid sequence as putative germline transformed plants.

2. The method of claim 1 wherein said plant expressible construct comprises a first nucleic acid sequence encoding a protein conferring a trait to said plant, said trait selected from the group consisting of genes that encode for insect tolerance, pest tolerance, herbicide resistance, quality enhancement, yield enhancement, stress tolerance, and environmental tolerance.

3. The method of claim 1 wherein said plant expressible construct comprises at least two nucleic acid sequences each encoding a protein.

4. The method of claim 3 wherein a first nucleic acid sequence encodes a protein conferring a trait to said plant and the second nucleic acid sequence encodes a protein permitting transformed plants to be selected in the presence of a corresponding selection agent.

5. The method of claim 4 wherein the roots are grown in the presence of a selection agent that corresponds to the protein.

6. The method of claim 5 wherein the selection agent is kanamycin.

7. The method of claim 5 wherein the selection agent is glyphosate.

8. The method of claim 1 wherein the transformation is via Agrobacterium-mediated or particle-mediated methods.

9. The method of claim 1 wherein the assay is an assay for a protein.

10. The method of claim 9, wherein the assay is selected from the group consisting of ELISA, colorometric methods, fluorometric methods, and enzymatic methods.

11. The method of claim 1 wherein the assay is an assay for a nucleic acid.

12. The method of claim 11 wherein the assay is selected from the group consisting of PCR, RT-PCR and Southern blots.

13. The method of claim 1 wherein the plant is a dicotyledonous plant.

14. The method of claim 13 wherein the plant is a soybean plant.

15. The method of claim 13 wherein the plant is a cotton plant.

16. A method of obtaining germline transformed plant cells via kanamycin selection comprising the steps of:
(a) providing a heterologous DNA construct comprising a promoter functional in plants operably connected to a DNA coding sequence, the coding sequence encoding a protein capable of conferring kanamycin resistance to a plant cell in which the sequence is expressed, and a 3″ termination sequence;
(b) inserting the DNA construct into the meristematic tissue of a plant embryo;
(c) inducing shoot formation from the treated meristematic tissue by culturing the treated meristematic tissue on a culture medium;
(d) culturing the shoots on a suitable shooting medium containing kanamycin at a concentration sufficient to significantly inhibit the growth of untransformed plant cells to generate kanamycin resistant shoots;
(e) regenerating the shoots of step (d) into genetically transformed plants having increased tolerance to kanamycin relative to wild type plants;
(f) assaying the roots from the plants of step (e) for the presence of the DNA construct; and
(g) identifying roots that assay positive for the DNA construct as putative germline transformed plants.

17. The method of claim 16, wherein the DNA construct additionally comprises a gene of interest.

18. The method of claim 16, wherein the DNA construct comprises the nptII gene.

19. The method of claim 16, wherein the DNA construct is inserted via Agrobacterium-mediated or particle-mediated methods.

20. A method for early identification of germline transformed plants comprising the steps of:
(a) transforming meristernatic or cotyledonary tissue with a plant expressible construct comprising at least one nucleic acid sequence encoding an antisense gene capable of affecting gene expression of an endogenous gene to obtain transformed plant tissue;
(b) producing a shoot from the transformed plant tissue;
(c) growing roots from the shoot;
(d) assaying the roots for the presence of the nucleic acid sequence; and
(e) identifying roots that assay positive for said at least one nucleic acid sequence as putative germline transformed plants.

21. The method of claim 20 wherein said plant expressible construct comprises at least two nucleic acid sequences, the first encodes an antisense gene and the second encodes a protein permitting transformed plants to be selected in the presence of a corresponding selection agent.
